(19) United States
(12) Patent Application Publication
(10) Pub. No.: US 2021/0030810 A1
(43) Pub. Date: Feb. 4, 2021

(54) COMPOSITIONS AND METHODS FOR TREATMENT OF SPINAL CORD INJURY

(71) Applicants: ANGIOCRINE BIOSCIENCE, INC., San Diego, CA (US); DREXEL UNIVERSITY, Philadelphia, PA (US)

(72) Inventors: Daniel Joseph Nolan, Hawthorne, NY (US); Michael Aron Lane, Philadelphia, PA (US); Liang Qiang, Philadelphia, PA (US); Lyandysha Viktorovna Zholudeva, Philadelphia, PA (US)

(21) Appl. No.: 16/963,897

(22) PCT Filed: Jan. 22, 2019
(86) PCT No.: PCT/US19/14497

§ 371 Date: Jul. 22, 2020

Related U.S. Application Data
(60) Provisional application No. 62/620,269, filed on Jan. 22, 2018.

(51) Int. Cl. A61K 35/30; A61K 38/16; A61K 35/76; A61P 25/00

(52) U.S. Cl. CPC A61K 35/30; A61P 25/00; A61K 35/76; A61K 38/162

(57) ABSTRACT

The present invention provides methods for treating spinal cord injury (SCI). Such methods involve administering E40RF1+ endothelial cells and neural cells (such as neural progenitor cells (NPCs), glial progenitor cells, or glial cells) to subjects having a SCI. The present invention also provides compositions useful in such methods, such as compositions comprising E40RF1+ endothelial cells and/or neural cells (such as NPCs, glial progenitor cells, or glial cells).

### CROSS-REFERENCE TO RELATED APPLICATIONS

This application claims the benefit of priority of U.S. Provisional Patent Application No. 62/620,269 filed on Jan. 22, 2018.

### INCORPORATION BY REFERENCE

For the purposes of those jurisdictions that permit incorporation-by-reference only, the text of all documents cited herein is hereby incorporated by reference in its entirety. In addition, any manufacturers' instructions or catalogues for any products cited or mentioned herein are incorporated by reference. Documents incorporated by reference into this text, or any teachings therein, can be used in the practice of the present invention. Many of the teachings of U.S. Pat. No. 8,465,732 entitled “Endothelial cells expressing adenovirus E4ORF1 and methods of use thereof” can be used in conjunction with the present invention or can be adapted for use with the present invention. Accordingly, the entire contents of U.S. Pat. No. 8,465,732 are hereby expressly incorporated by reference into the present application.

### BACKGROUND OF THE INVENTION

Spinal cord injury (“SCI”) results in a vast range of debilitating and potentially life-threatening deficits. For example, SCI at the cervical (neck) level frequently results in life-threatening respiratory deficits, which can be attributed in large part to the direct compromise of the phrenic motor circuitry that controls the diaphragm—the primary respiratory muscle. Other devastating effects of SCI include paraplegia and quadriplegia. With more than 250,000 incidents of SCI occurring world-wide each year, there is an urgent need to develop treatments capable of improving survival, function, and quality of life in affected individuals.

Cell therapies are among some of the most promising treatment strategies being explored—with the ultimate goal being achieving functional recovery from SCI. Several studies have investigated the safety and efficacy of transplanting neural progenitor cells (“NPCs”) into the injured spinal cord. NPCs represent an extensively investigated source of transplantable, lineage-restricted (neuronal and glial) precursors that retain a proliferative capacity. However, to date, pre-clinical studies using NPCs to treat spinal cord injury have yielded variable outcomes (1). Angiogenesis is thought to be an essential component of tissue repair. However, surprisingly little effort has been made to enhance angiogenesis/vasculogenesis in the context of SCI. Some studies have explored the use of viral vectors to supply vascular growth factors (e.g. VEGF and FGF), and while associated with some repair potential (34), a significant limitation with these approaches is that they are non-biological and thus the dose and time course of trophic factor delivery remain unregulated. A recent study reported that transplantation of a degradable polymer implant containing naïve endothelial cells (“ECs”) together with NPCs at the site of a spinal cord lesion could promote the formation of stable functional blood vessels at the site of the rat spinal cord lesions (Rauch et al., 2009). However, while some sprouting of neurofilament-positive cells was seen, no evidence of the growth/extension of neurons and/or axons spanning the lesion was reported, and no recovery of neural function was reported (Rauch et al., 2009). Given the lack of success of current cell therapy efforts, there remains a need in the art for robust and effective cellular therapies capable of achieving functional spinal cord repair. The present invention addresses this need.

### SUMMARY OF THE INVENTION

The present invention derives, in part, from several surprising discoveries that are described in more detail in the Examples section of this patent specification. In particular, it has now been discovered that transplantation of neural cells together with engineered endothelial cells that express an adenovirus E4ORF1 sequence (“E4ORF1⁺ ECs”) at the site of a spinal cord lesion results in a dramatic and unexpected level of neural repair characterized by growth/extension of axons through the spinal cord lesion and, importantly, recovery from SCI-associated functional deficits—impaired diaphragm function and breathing. Building on these findings, and other findings described in the Examples section of this patent specification, the present invention provides various new and improved compositions and methods for use in the repair of spinal cord injury.

Accordingly, in some embodiments the present invention provides methods of treating spinal cord injury (SCI) in a subject in need thereof, the methods comprising: administering: (a) E4ORF1+ endothelial cells (ECs), and (b) neural cells, to a subject having a SCI, for example by local administration at the site of the SCI, thereby treating the SCI in the subject. Similarly, in other embodiments, the present invention provides compositions comprising (a) E4ORF1+ endothelial cells (ECs), and (b) neural cells. Such compositions may be useful in treating SCI in subjects in need thereof.

One important feature of the methods and compositions described herein is their ability to generate meaningful anatomical and functional neural repair. In some embodiments the “treatment” achieved using the methods and compositions of the invention comprises neural repair. In some embodiments the “treatment” achieved using the methods and compositions of the invention comprises growth and/or extension of neurons and/or axons through a spinal cord lesion. In some embodiments the “treatment” achieved using the methods and compositions of the invention comprises growth and/or extension of motor neurons and/or axons through a spinal cord lesion. In some embodiments the “treatment” achieved using the methods and compositions of the invention comprises growth and/or extension of sensory neurons and/or axons through a spinal cord lesion. In some embodiments the “treatment” achieved using the methods and compositions of the invention comprises growth and/or extension of serotonergic neurons and/or axons through a spinal cord lesion. In some embodiments the “treatment” achieved using the methods and compositions of the invention comprises growth and/or extension of phrenic neurons and/or axons through a spinal cord lesion. In some embodiments the “treatment” achieved using the methods and compositions of the invention comprises growth and/or extension of neurons and/or axons through a spinal cord lesion wherein the neurons and/or axons become synaptically integrated into the subject's central nervous system. In some embodiments the “treatment” achieved using the methods and compositions of the invention comprises an increase in transmittal of electrical signals across a spinal cord lesion. In some embodiments the “treatment” achieved using the methods and compositions of the invention comprises an improvement in a motor function that was compromised or lost as the result of the spinal cord lesion. In some embodiments the “treatment” achieved using the methods and compositions of the invention comprises an improvement in a sensory function that was compromised or lost as the result of the spinal cord lesion. In some embodiments the “treatment” achieved using the methods and compositions of the invention comprises an improvement in diaphragm function and/or breathing.

In each of such methods and compositions, various different types of ECs can be used. For example, in some embodiments the ECs are vascular ECs. In some embodiments the ECs are primary ECs, while in other embodiments the ECs are cultured EC cells from an EC cell line. In some embodiments the ECs are mammalian ECs. In some embodiments the ECs are primate ECs. In some embodiments the ECs are human ECs. In some embodiments the ECs are other mammalian ECs, such as rabbit, rat, mouse, guinea pig, goat, pig, sheep, cow, horse, cat or dog ECs. In some embodiments the ECs are umbilical vein ECs (UVECs). In some embodiments the ECs are human umbilical vein ECs (HUVECs). In some embodiments the ECs are central-nervous system ECs. In some embodiments the ECs are brain ECs. In some embodiments the ECs are spinal cord ECs. In some embodiments the ECs are olfactory bulb ECs. In some embodiments the ECs are peripheral-nervous system ECs. In some embodiments the ECs are allogeneic with respect to the subject into which they are to be transplanted/administered. In some embodiments the ECs are autologous with respect to the subject into which they are to be transplanted/administered. In some embodiments the ECs have the same MHC/HLA type as the subject into which they are to be transplanted/administered. In some embodiments the ECs are mitotically inactive. In some embodiments the ECs are differentiated ECs. In some embodiments the ECs are adult ECs. In some embodiments the ECs are differentiated from induced pluripotent stem cells (iPSCs). In some embodiments the ECs are differentiated from iPSCs induced from cells including, but not limited to, skin, fibroblasts, hepatocytes, lymphoblasts, astrocytes, peripheral blood mononuclear cells. In some embodiments the ECs are produced by trans-differentiation from a differentiated non-endothelial cell type. In some embodiments the ECs have been previously cultured in a 3D matrix. In some embodiments the ECs have not been previously cultured in a 3D matrix.

Similarly, in each of such methods and compositions, various different types of neural cells can be used. For example, in some embodiments the neural cells are primary neural cells. In some embodiments the neural cells are cultured from a neural cell line or from a primary source. In some embodiments the neural cells are mammalian neural cells. In some embodiments the neural cells are primate neural cells. In some embodiments the neural cells are human neural cells. In some embodiments the neural cells are other mammalian cells, such as rabbit, rat, mouse, guinea pig, goat, pig, sheep, cow, horse, cat or dog neural cells. In some embodiments the neural cells are neuronal cells. In some embodiments the neural cells are glial cells. In some embodiments the neural cells are neural stem cells (NSCs). In some embodiments the neural cells are neural progenitor cells (NPCs). In some embodiments the neural cells are neural progenitor cells (NPCs) derived from the spinal cord. In some embodiments the neural cells are neural progenitor cells (NPCs) derived from the olfactory bulb. In some embodiments the neural cells are neural progenitor cells (NPCs) derived from the spinal cord or the olfactory bulb. In some embodiments the neural cells are neural progenitor cells (NPCs) derived from the developing spinal cord. In some embodiments the neural cells are neural progenitor cells (NPCs) derived from the developing olfactory bulb. In some embodiments the neural cells are neural progenitor cells (NPCs) derived from the developing spinal cord or the developing olfactory bulb. In some embodiments the neural cells are lineage-restricted neuronal progenitor cells or glial progenitor cells. In some embodiments the neural cells are allogeneic with respect to the subject into which they are to be transplanted/administered. In some embodiments the neural cells are autologous with respect to the subject into which they are to be transplanted/administered. In some embodiments the neural cells have the same MHC/HLA type as the subject into which they are to be transplanted/administered. In some embodiments the neural cells are mitotically inactive. In some embodiments the neural cells are differentiated neural cells. In some embodiments the neural cells are adult neural cells. In some embodiments the neural cells are differentiated from induced pluripotent stem cells (iPSCs). In some embodiments the neural cells are differentiated from iPSCs induced from cells including, but not limited to, skin, fibroblasts, hepatocytes, lymphoblasts, astrocytes, peripheral blood mononuclear cells. In some embodiments the neural cells are produced by trans-differentiation from a differentiated non-neural cell type. In some embodiments the neural cells have been previously cultured in a 3D matrix. In some embodiments the neural cells have not been previously cultured in a 3D matrix.

The subjects that can be treated with the methods and compositions of the present invention include any subjects having a spinal cord injury (SCI). In some embodiments the subject is a mammal. In some embodiments the subject is a primate. In some embodiments the subject is a human. In some embodiments the subject is a rabbit, rat, mouse, guinea pig, goat, pig, sheep, cow, horse, cat or dog.

Each of the methods and compositions of the invention involves endothelial cells that contain an adenovirus E4ORF1 polypeptide—i.e. “E4ORF1+ ECs.” In some such embodiments the E4ORF1+ ECs contain a nucleic acid molecule that encodes an adenovirus E4ORF1 polypeptide. In some embodiments that nucleic acid molecule is present in a vector. In some embodiments the vector is a retroviral vector. In some embodiments the retroviral vector is a lentiviral vector. In some embodiments the retroviral vector is a Maloney murine leukemia virus (MMLV) vector. In some embodiments the nucleic acid that encodes the adenovirus E4ORF1 polypeptide is integrated into the genomic DNA of the ECs.

In carrying out the treatment methods of the present invention, the E4ORF1+ ECs and/or neural cells can be administered using any suitable means known in the art for local delivery of cells or agents to the site of a spinal cord injury. In some embodiments the E4ORF1+ ECs and/or neural cells are administered by local injection. In some embodiments the E4ORF1+ ECs and/or neural cells are administered by local infusion. In some embodiments the E4ORF1+ ECs and/or neural cells are administered by local surgical implantation methods. In some embodiments the E4ORF1+ ECs and/or neural cells are administered in a biocompatible matrix material (e.g. a biocompatible and/or biodegradable matrix, such as a solid 3D implant or a liquid matrix). In some embodiments the E4ORF1+ ECs and/or neural cells are not administered in a biocompatible matrix material. In some embodiments the E4ORF1+ ECs and/or neural cells are not administered in a solid 3D biocompatible matrix. Similarly, the E4ORF1+ ECs and/or neural cells can be administered in any suitable carrier composition known in the art. For example, in some embodiments the cells may be administered in a composition comprising a physiological saline. In some embodiments the cells may be administered in a biocompatible matrix material—such as one that remains liquid during the administration process, or one that is a solid 3D implant during the administration process. The E4ORF1+ ECs and/or neural cells can be administered together or separately. The E4ORF1+ ECs and/or neural cells can also be administered concurrently or at different times. In some embodiments the E4ORF1+ ECs and/or neural cells will be administered to the subject only once, while in other embodiments the E4ORF1+ ECs and/or neural cells may be administered to the subject multiple times. The ratio of the E4ORF1+ ECs to neural cells that is administered can be varied. In some embodiments a 1:1 ratio of E4ORF1+ ECs to neural cells is used. However, E4ORF1+ EC to neural cell ratios of about 1:10, or about 1:9, or about 1:8, or about 1:7, or about 1:6, or about 1:5, or about 1:4, or about 1:3, or about 1:2, or about 2:1, or about 3:1, or about 4:1, or about 5:1, or about 6:1, or about 7:1, or about 8:1, or about 9:1, or about 10:1, can also be used. Similarly, the numbers of E4ORF1+ ECs and neural cells that are administered can also be varied. The number of E4ORF1+ ECs administered should be an “effective amount” as defined herein. Similarly, the number of neural cells administered should be an “effective amount” as defined herein. In some embodiments the total number of cells that is administered is in the range of about 500,000 cells to about 10,000,000 (10 million) cells. In some embodiments, such as those where cells are administered to small animals such as rodents, the total number of cells that is administered is in the range of about 500,000 cells to about 2,000,000 (2 million) cells. In some embodiments, such as those where cells are administered to larger animals such as primates (including humans), the total number of cells that is administered is in the range of about 5,000,000 (5 million) cells to about 10,000,000 (10 million) cells. After the E4ORF1+ ECs and the neural cells have been administered, the progress of the treatment can be monitored at various times (for example, beginning with immediate assessment, continuing on a daily basis for the first week and, twice a week thereafter for an indefinite amount of time or up to completion of pre-set experimental time-point) using various different methods. Example of such methods include, but are not limited to, methods that enable anatomical repair to be visualized (e.g. using medical imaging techniques, or histological assessment when appropriate), and methods that enable functional improvements to be observed (e.g. by measuring one or more sensory or motor functions affected by the SCI).

In carrying out the treatment methods of the present invention, the timing of the administration of the E4ORF1+ ECs and/or the neural cells to the subject can be any suitable time after the creation of the injury. In the case of human subjects, a physician will typically make a determination about the timing of the administration. In some embodiments, the E4ORF1+ ECs and/or the neural cells are administered to the subject within the acute phase after the creation of the SCI injury. In some embodiments, the E4ORF1+ ECs and/or the neural cells are administered to the subject within the subacute phase after the creation of the SCI injury. In some embodiments, the E4ORF1+ ECs and/or the neural cells are administered to the subject within the intermediate phase after the creation of the SCI injury. In some embodiments, the E4ORF1+ ECs and/or the neural cells are administered to the subject within the chronic phase after the creation of the SCI injury. In some embodiments, the E4ORF1+ ECs and/or the neural cells are administered to the subject within about 1 week of the creation of the SCI injury. In some embodiments, the E4ORF1+ ECs and/or the neural cells are administered to the subject within about 2 week2 of the creation of the SCI injury. In some embodiments, the E4ORF1+ ECs and/or the neural cells are administered to the subject within about 3 weeks of the creation of the SCI injury. In some embodiments, the E4ORF1+ ECs and/or the neural cells are administered to the subject within about 4 weeks of the creation of the SCI injury.

These and other embodiments of the invention are described further in the other sections of this patent disclosure. In addition, as will be apparent to those of skill in the art, certain modifications and combinations of the various embodiments described herein fall within the scope of the present invention.

### BRIEF DESCRIPTION OF THE DRAWINGS

FIG. 1A-E. Schematic diagrams of methods and timelines employed in the experiments described in the Examples section of this patent specification. FIG. 1A—Schematic diagram of NPC isolation (from developing spinal cord), culture, freezing, storage and thawing prior to transplantation. FIG. 1B—Schematic diagram of EC isolation (from spinal cord), selection and culture prior to transplantation. FIG. 1C—Schematic diagram of combined NPC and EC transplantation at the site of a spinal cord injury by injection. The diagram also illustrates anterograde and retrograde tracing methods. FIG. 1D—Schematic diagram of typical experimental timeline. FIG. 1E—Additional schematic diagram of the SCI injury model used in the experiments described in Examples 1-3. The left-hand panel shows the cervical spinal cord and the anatomy of the phrenic motor circuit following a lateralized cervical (C) 3/4 contusion injury. Inspiratory neurons in the ventral respiratory column (VRC) (i) innervate phrenic motoneurons (ii) and spinal interneurons (SpINs; iii). Contusive injury (iv) disrupts both white and grey matter, denervating the motor pool caudal to injury (v). The right-hand panel shows schematically the injection of endothelial cells (ECs) and neural progenitor cells (NPC) into the contusion cavity (vi), for example 1 week post-injury.

FIG. 2 A-C. Results of phenotypic analysis of transplanted NPCs and ECs showing differentiation into glial fibrillary acidic protein (GFAP) positive glia 6 weeks after transplantation, as described in more detail in the Examples section of this patent specification. Transplanting GFP-expressing NPCs and ECs (FIG. 2A) results in high expression of GFAP positive glia (FIG. 2B) 6 weeks after transplantation. FIG. 2C shows a scatter plot used for calculating the Manders colocalization coefficient, where Quadrant 1 (Q1) represents pixels that have high GFAP intensities and low GFP intensities; Q2 represents pixels with high intensity levels in both GFAP and GFP channels and Q4 represents high GFP and low GFAP intensities. Q3 represents pixels that have low intensity levels in both channels. This assessment revealed an average Manders coefficient of 0.96 (N=3).

FIG. 3 A-D. Results of transplantation of NPCs and ECs showing enhanced serotonergic (5HT-positive) growth through the lesion cavity, as described in more detail in the Examples section of this patent specification. Transplantation of NPCs with endothelial cells (ECs) results in enhanced serotonergic growth through the lesion cavity. Transplanted GFP labeled NPCs and ECs survive 6 weeks after transplantation (FIG. 3A), yield GFAP positive glia (FIG. 3B) and result in increased vascularization throughout the lesion cavity as depicted by Rat Endothelial Cell Antigen (RECA) staining (FIG. 3C). The combinatorial transplant (NPCs+ECs) results in host serotonergic (5HT) growth through the lesion cavity (FIG. 3D). In each of FIG. 3A-D, the white arrows show growing axons. Scale bars are as indicated.

FIG. 4. Results of transplantation of NPCs and ECs showing recovery of diaphragm function 6 weeks after transplantation, as described in more detail in the Examples section of this patent specification. Diaphragm function was assessed 6 weeks after transplantation using terminal diaphragm electromyography (dEMGs) during baseline (normal breathing) and under a respiratory challenge (hypoxia, 10% O2). The percent change (i.e. the animal's ability to respond to the respiratory challenge) is represented with each dot being an average of a 40 second recording from each animal. The bar graphs represent the average of each indicated group.

### DETAILED DESCRIPTION

The “Summary of the Invention,” “Figures,” “Brief Description of the Figures,” “Examples,” and “Claims” sections of this patent disclosure describe the main embodiments of the invention. This “Detailed Description” section provides certain additional description relating to the compositions and methods of the present invention, and is intended to be read in conjunction with all other sections of this patent disclosure. Furthermore, and as will be apparent to those in the art, the different embodiments described throughout this patent disclosure can be, and are intended to be, combined in various different ways. Such combinations of the specific embodiments described herein are intended to fall within the scope of the present invention.

### Definitions & Abbreviations

Certain definitions and abbreviations are provided below. Others may be defined elsewhere in this patent disclosure. Furthermore, unless defined otherwise, all technical and scientific terms and abbreviations used herein have the same meaning as commonly understood by one of ordinary skill in the art to which this invention is related. For example, The Dictionary of Cell and Molecular Biology (5th ed. J. M. Lackie ed., 2013), the Oxford Dictionary of Biochemistry and Molecular Biology (2d ed. R. Cammack et al. eds., 2008), and The Concise Dictionary of Biomedicine and Molecular Biology (2d ed. P-S. Juo, 2002) can provide one of skill with general definitions of some terms used herein.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Système International de Unites (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10.

Wherever embodiments are described with the language “comprising,” otherwise analogous embodiments described in terms of “consisting of” and/or “consisting essentially of” are included.

As used herein, the terms “about” and “approximately,” when used in relation to numerical values, mean within + or −10% of the stated value.

As used herein, the term “allogeneic” means deriving from, originating in, or being members of the same species, where the members are genetically related or genetically unrelated but genetically similar. An “allogeneic transplant” refers to transfer of cells from a donor subject to a recipient subject, where the recipient subject is the same species as the donor subject. In some allogeneic transplantation methods, the donor subject and the recipient subject have the same MHC/HLA type—i.e. the donor subject and the recipient subject are MHC-matched or HLA-matched. In some allogeneic transplantation embodiments, cells are: (a) obtained from a first/donor subject, (b) optionally maintained and/or cultured and/or expanded and/or modified ex vivo, and (c) subsequently transplanted into a second/recipient subject of the same species as the first/donor subject. For example, in some allogeneic transplantation embodiments, endothelial cells are obtained from a first/donor subject, genetically modified ex vivo to render them E4ORF1+, and then transplanted into a second/recipient subject of the same species as the first/donor subject.

As used herein, the term “autologous” means deriving from or originating in the same subject. An “autologous transplant” refers to administration of a subject's own cells to the subject—i.e. in the case of autologous transplantation, the “donor” and the “recipient” of the transplanted cells are the same individual. In some autologous transplantation embodiments, cells are: (a) obtained from a subject, (b) optionally maintained and/or cultured and/or expanded and/or modified ex vivo, and (c) subsequently transplanted back into the same subject. For example, in some autologous transplantation embodiments, endothelial cells are obtained from a subject, genetically modified ex vivo to render them E4ORF1+, and then transplanted back into the same subject.

As used herein, the abbreviation “EC” refers to an endothelial cell.

As used herein, the abbreviation “E4ORF1” refers to open reading frame 1 of the early 4 region of an adenovirus genome.

As used herein the term “effective amount” refers to an amount of a specified agent or cell population (e.g. an E4ORF1 polypeptide, a nucleic acid molecule encoding an E4ORF1 polypeptide, or a population of E4ORF1+ engineered endothelial cells or a population of neural cells), as described herein, that is sufficient to achieve the stated purpose described herein. For example, in the case of expression of E4ORF1 in endothelial cells an effective amount of a nucleic acid molecule (e.g. in a vector) to be introduced/delivered to the endothelial cells may be one that results in detectable expression of E4ORF1 protein in the endothelial cells. In the case of methods that involve administering E4ORF1⁺ endothelial cells and/or neural cells to a subject, an effective amount of such cells or cell combinations may be one that results in a detectable degree of, or a detectable improvement of, one or more indicators of SCI repair, including, but not limited to, growth of axons through or around a SCI lesion and recovery of sensory or motor function in one or more sensory or motor systems. An appropriate “effective amount” in any individual case may be determined empirically, for example using standard techniques known in the art, such as dose or cell number escalation studies, and may be determined considering such factors as the planned use, the planned mode of delivery/administration, desired frequency of delivery/administration, whether one, two, or more cell types are to be delivered/administered, etc. Furthermore, an “effective amount” may be determined using assays such as those described in the Examples section of this patent disclosure to assess effects on SCI repair. Such assays include, but are not limited to, those based on studying anatomical indicators of SCI repair and those based on studying functional indicators of SCI repair.

The term “engineered” when used in relation to cells herein refers to cells that have been engineered by man to result in the specified phenotype (e.g. E4ORF1V), or to express a specified nucleic acid molecule or polypeptide. The term “engineered cells” is not intended to encompass naturally occurring cells, but is, instead, intended to encompass, for example, cells that comprise a recombinant nucleic acid molecule, or cells that have otherwise been altered artificially (e.g. by genetic modification), for example so that they express a polypeptide that they would not otherwise express, or so that they express a polypeptide at substantially higher levels than that observed in non-engineered endothelial cells.

As used herein the term “isolated” refers to a product, compound, composition, or cell population (including a population of one cell type or of multiple specified cell types) that is separated from at least one other product, compound, composition or cell population with which it is associated in its naturally occurring state, whether in nature or as made synthetically.

As used herein, the abbreviation “NPC” refers to a neural progenitor cell. As used herein, the abbreviation “NSC” refers to a neural stem cell.

As used herein, the term “recombinant” refers to nucleic acid molecules that are generated by man (including by a machine) using methods of molecular biology and genetic engineering (such as molecular cloning), and that comprise nucleotide sequences that would not otherwise exist in nature. Thus, recombinant nucleic acid molecules are to be distinguished from nucleic acid molecules that exist in nature—for example in the genome of an organism. A nucleic acid molecule that comprises a complementary DNA or “cDNA” copy of an mRNA sequence, without any intervening intronic sequences such as would be found in the corresponding genomic DNA sequence, would thus be considered a recombinant nucleic acid molecule. By way of example, a recombinant E4ORF1 nucleic acid molecule might comprise an E4ORF1 coding sequence operatively linked to a promoter and/or other genetic elements with which that coding sequence is not ordinarily associated in a naturally-occurring adenovirus genome.

The term “subject” refers to, except where indicated, mammals such as humans and non-human primates, as well as rabbits, rats, mice, goats, pigs, and other mammalian species to be treated using the compositions or methods described herein.

The phrase “substantially pure” as used herein in relation to a cell population refers to a population of cells of a specified type (e.g. as determined by expression of one or more specified cell markers, morphological characteristics, or functional characteristics), or of specified types (plural) in embodiments where two or more different cell types are used together, that is at least about 50%, preferably at least about 75-80%, more preferably at least about 85-90%, and most preferably at least about 95% of the cells making up the total cell population. Thus, a “substantially pure cell population” refers to a population of cells that contain fewer than about 50%, preferably fewer than about 20-25%, more preferably fewer than about 10-15%, and most preferably fewer than about 5% of cells that are not of the specified type or types.

Terms such as “treating” or “treatment” or “to treat” refer to measures that detectably cure, reverse, slow down, lessen symptoms of, or improve symptoms of, and/or halt progression of a specified condition or disorder (such as SCI) and/or that result in either a detectable improvement in an injury (such as SCI) at either an anatomical level, a functional level, or both—in a subject. In certain embodiments, a subject is successfully “treated” according to the methods provided herein if the subject shows, e.g., a total or partial, and/or a permanent or transient, alleviation or elimination of an injury or symptoms of an injury—such as SCI. Thus, successful “treatment” using the methods of the present invention may include, but is not limited to, an increase in axon projections around or across a spinal cord lesion, and/or an increase in transmittal of electrical signals across a spinal cord lesion, and/or an improvement in a function that was previously compromised or lost as the result of a spinal cord lesion (such as a motor function or sensory function), where such increases or improvements may be partial, total, transient, or permanent.

### E4ORF1 Nucleic Acid Molecules and Polypeptides

The present invention involves E4ORF1+ ECs. E4ORF1+ ECs are endothelial cells that comprise an adenovirus E4ORF1 polypeptide, which is typically encoded by an E4ORF1 nucleic acid molecule. In some embodiments the present invention involves E4ORF1 polypeptides and/or nucleic acid molecules that encode an adenovirus E4ORF1 polypeptide.

The adenoviral early 4 (E4) region contains at least 6 open reading frames (E4ORFs). The entire E4 region has been shown previously to promote survival of endothelial cells (see Zhang et al. (2004), J. Biol. Chem. 279(12):11760-66). It has also been shown previously that, within the entire E4 region, it is the E4ORF1 sequence that is responsible for these biological effects in endothelial cells. See U.S. Pat. No. 8,465,732. See also Seandel et al. (2008), “Generation of a functional and durable vascular niche by the adenoviral E4ORF1 gene,” PNAS, 105(49):19288-93.

The E4ORF1 polypeptides of the invention and the E4ORF1 nucleic acid molecules of the invention may have amino acid sequences or nucleotide sequences that are specified herein or known in the art, or may have amino acid or nucleotide sequences that are variants, derivatives, mutants, or fragments of such amino acid or nucleotide sequences—provided that such a variants, derivatives, mutants, or fragments have, or encode a polypeptide that has, one or more of the functional properties of E4ORF1 described in U.S. Pat. No. 8,465,732 or described herein, including, but not limited to, those associated with EC function and/or SCI repair.

In those embodiments involving E4ORF1 polypeptides, the polypeptide sequence used may be from any suitable adenovirus type or strain, such as human adenovirus type 2, 3, 5, 7, 9, 11, 12, 14, 34, 35, 46, 50, or 52. In some embodiments the polypeptide sequence used is from human adenovirus type 5. Amino acid sequences of such adenovirus polypeptides, and nucleic acid sequences that encode such polypeptides, are well known in the art and available in well-known publicly available databases, such as the Genbank database. For example, suitable sequences include the following: human adenovirus 9 (Genbank Accession No. CAI05991), human adenovirus 7 (Genbank Accession No. AAR89977), human adenovirus 46 (Genbank Accession No. AAX70946), human adenovirus 52 (Genbank Accession No. ABK35065), human adenovirus 34 (Genbank Accession No. AAW33508), human adenovirus 14 (Genbank Accession No. AAW33146), human adenovirus 50 (Genbank Accession No. AAW33554), human adenovirus 2 (Genbank Accession No. AP.sub.-000196), human adenovirus 12 (Genbank Accession No. AP.sub.-000141), human adenovirus 35 (Genbank Accession No. AP.sub.-000607), human adenovirus 7 (Genbank Accession No. AP.sub.-000570), human adenovirus 1 (Genbank Accession No. AP.sub.-000533), human adenovirus 11 (Genbank Accession No. AP.sub.-000474), human adenovirus 3 (Genbank Accession No. ABB 17792), and human adenovirus type 5 (Genbank accession number D12587).

In some embodiments, the E4ORF1 polypeptides and/or E4ORF1 nucleic acid molecules used in accordance with the present invention have the same amino acid or nucleotide sequences as those specifically recited herein or known in the art (for example in public sequence databases, such as the Genbank database). In some embodiments the E4ORF1 polypeptides and E4ORF1 nucleic acid molecules used may have amino acid or nucleotide sequences that are variants, derivatives, mutants, or fragments of such sequences, for example variants, derivatives, mutants, or fragments having greater than 85% sequence identity to such sequences. In some embodiments, the variants, derivatives, mutants, or fragments have about an 85% identity to the known sequence, or about an 88%, 89%, 90%, 91%, 92%, 93%, 94%, 95%, 96%, 97%, 98%, or 99% sequence identity to the known sequence. In some embodiments, a variant, derivative, mutant, or fragment of a known E4ORF1 nucleotide sequence is used that varies in length by about 50 nucleotides, or about 45 nucleotides, or about 40 nucleotides, or about 35 nucleotides, or about 30 nucleotides, or about 28 nucleotides, 26 nucleotides, 24 nucleotides, 22 nucleotides, 20 nucleotides, 18 nucleotides, 16 nucleotides, 14 nucleotides, 12 nucleotides, 10 nucleotides, 9 nucleotides, 8 nucleotides, 7 nucleotides, 6 nucleotides, 5 nucleotides, 4 nucleotides, 3 nucleotides, 2 nucleotides, or 1 nucleotide relative to the known E4ORF1 nucleotide sequence. In some embodiments, a variant, derivative, mutant, or fragment of a known E4ORF1 amino sequence is used that varies in length about 50 amino acids, or about 45 amino acids, or about 40 amino acids, or about 35 amino acids, or about 30 amino acids, or about 28 amino acids, 26 amino acids, 24 amino acids, 22 amino acids, 20 amino acids, 18 amino acids, 16 amino acids, 14 amino acids, 12 amino acids, 10 amino acids, 9 amino acids, 8 amino acids, 7 amino acids, 6 amino acids, 5 amino acids, 4 amino acids, 3 amino acids, 2 amino acids, or 1 amino acid relative to the known E4ORF1 amino acid sequence.

Nucleic acid molecules that encode E4ORF1 may comprise naturally occurring nucleotides, synthetic nucleotides, or a combination thereof. For example, in some embodiments nucleic acid molecules that encode E4ORF1 can comprise RNA, such as synthetic modified RNA that is stable within cells and can be used to direct protein expression/production directly within cells. In other embodiments the nucleic acid molecules that encode E4ORF1 can comprise DNA.

In some embodiments E4ORF1 sequences are used without other sequences from an adenovirus E4 region—for example not in the context of the nucleotide sequence of the entire E4 region or not together with other polypeptides encoded by the E4 region. However, in some other embodiments E4ORF1 sequences may be used in conjunction with one or more other nucleic acid or amino acid sequences from an adenovirus E4 region, such as E4ORF2, E4ORF3, E4ORF4, E4ORF5 or E4ORF6 sequences, or variants, mutants or fragments thereof. For example, although E4ORF1 sequences can be used in constructs (such as a viral vectors) that contain other sequences, genes, or coding regions (such as promoters, marker genes, antibiotic resistance genes, and the like), in certain embodiments, the E4ORF1 sequences are used in constructs that do not contain an entire adenovirus E4 region, or that do not contain other ORFs from an adenovirus E4 region, such as E4ORF2, E4ORF3, E4ORF4, E4ORF5 and/or E4ORF6.

Nucleic acid sequence that encode E4ORF1 will typically be provided in a vector. Similarly, E4ORF1+ ECs will typically contain a vector—i.e. a vector containing a nucleic acid sequence that encodes E4ORF1. The term “vector” is used in accordance with its usual meaning in the art, and includes, for example, a tool that can be used for the transfer of a nucleic acid molecule (such as a nucleic acid molecule that encodes E4ORF1) into a cell, such as an endothelial cell. The term “vector” as used herein includes: vectors that serve to maintain a nucleic acid molecule in a cell, vectors that can replicate within a cell, vectors that cannot replicate within a cell, vectors that can incorporate into the genome of a cell (integrating vectors), vectors that do not incorporate into the genome of a cell (non-integrating vectors), and vectors that allow expression of a polypeptide encoded by a nucleic acid molecule within the vector—i.e. expression vectors. The term “vector” as used herein also includes both viral vectors and non-viral vectors. Viral vectors include, but are not limited to, those derived from retroviruses, adenoviruses, adeno-associated viruses, herpes simplex viruses, vaccinia viruses and baculoviruses. Examples of retroviral vectors include, but are not limited to, those derived from a lentivirus (e.g. HIV-1, HIV-2, SIV, FIV, BIV, EIAV, CAEV or visna lentivirus), a murine leukemia virus (MLV), a human T-cell leukemia virus (HTLV), a mouse mammary tumour virus (MMTV), a Rous sarcoma virus (RSV), a Fujinami sarcoma virus (FuSV), a Moloney murine leukemia virus (MMLV or MoMLV), a FBR murine osteosarcoma virus (FBR MSV), a Moloney murine sarcoma virus (Mo-MSV), an Abelson murine leukemia virus (A-MLV), an Avian myelocytomatosis virus-29 (MC29) or an Avian erythroblastosis virus (AEV). Furthermore, a detailed list of retroviruses may be found in Coffin et al. (1997) (“Retroviruses”, Cold Spring Harbour Laboratory Press Eds: J M Coffin, S M Hughes, H E Varmus pp 758-763), and retroviral vectors may be derived from such other retroviruses. Unlike most retroviruses, lentiviruses have can infect both dividing and non-dividing cells (Lewis et al (1992) EMBO J 11(8):3053-3058 and Lewis and Emerman (1994) J Virol 68 (1):510-516). This is in contrast to most other retroviruses, which infect dividing/mitotic cells.

According to the present invention, a nucleic acid sequence that encodes E4ORF1 may be provided in any suitable vector, such as any suitable vector from those described above. Similarly, according to the present invention E4ORF1+ ECs may comprise any such suitable vector. In some embodiments a retroviral vector, such as a lentiviral vector or an MMLV vector, is used. However, one of ordinary skill in the art will be able to select other suitable vectors. Typically, the vector will be an expression vector suitable for transfection/transduction of endothelial cells and suitable for expression of E4ORF1 in endothelial cells. In such expression vectors the nucleic acid sequence that encodes E4ORF1 will be operatively linked to one or more promoters to allow for expression. Any promoter suitable to drive expression of the E4ORF1 nucleic acid sequence in the desired endothelial cell type can be used. Examples of suitable promoters include, but are not limited to, the CMV, SV40, RSV, HIV-Ltr, and MML promoters. The promoter can also be a promoter from the adenovirus genome, or a variant thereof. For example, the promoter can be a promoter that drives E4ORF1 expression in an adenovirus genome. In some embodiments an inducible/regulatable promoter may be used, so that expression can be turned on or off as desired. Any suitable inducible or regulatable expression system can be used, such as, for example, a tetracycline inducible expression system, or a hormone inducible expression system. In addition to containing nucleic acid sequences that encode E4ORF1, the vectors used may also contain various other nucleic acid sequences, genes, or coding regions, depending on the desired use, for example, antibiotic resistance genes, reporter genes or expression tags (such as, for example nucleotides sequences encoding GFP), or any other nucleotide sequences or genes that might be desirable. E4ORF1 polypeptides can be expressed alone or as part of fusion proteins.

Nucleic acid molecules that encode E4ORF1, and vectors that comprise such nucleic acid molecules, can be introduced into endothelial cells using any suitable system known in the art, including, but not limited to, transfection techniques and viral-mediated transduction techniques. Transfection methods that can be used in accordance with the present invention include, but are not limited to, liposome-mediated transfection, polybrene-mediated transfection, DEAE dextran-mediated transfection, electroporation, calcium phosphate precipitation, microinjection, and micro-particle bombardment. Viral-mediated transduction methods that can be used include, but are not limited to, lentivirus-mediated transduction, adenovirus-mediated transduction, retrovirus-mediated transduction, adeno-associated virus-mediated transduction, vaccinia virus-mediated transduction, and herpesvirus-mediated transduction.

In some embodiments an E4ORF1 peptidomimetic may be used. A peptidomimetic is a small protein-like chain designed to mimic a polypeptide. Such a molecule could be designed to mimic an E4ORF1 polypeptide. Various different ways of modifying a peptide to create a peptidomimetic, or otherwise designing a peptidomimetic, are known in the art and can be used to create an E4ORF1 peptidomimetic for use in the methods of the present invention.

The handling, manipulation, and expression of E4ORF1 polypeptides and/or E4ORF1 nucleic acid molecules may be performed using conventional techniques of molecular biology and cell biology. Such techniques are well known in the art. For example, one may refer to the teachings of Sambrook, Fritsch and Maniatis eds., “Molecular Cloning A Laboratory Manual, 2nd Ed., Cold Springs Harbor Laboratory Press, 1989); the series Methods of Enzymology (Academic Press, Inc.), or any other standard texts for guidance on suitable techniques to use in handling, manipulating, and expressing nucleotide and/or amino acid sequences. Additional aspects relevant to the handling or expression of E4ORF1 amino acid and nucleotide sequences are described in U.S. Pat. No. 8,465,732, the contents of which are hereby incorporated by reference.

### Endothelial Cells

The present invention involves E4ORF1+ ECs, compositions that comprise E4ORF1+ ECs, and methods of use of such E4ORF1+ ECs and compositions.

The ECs can be, or can be derived from, any type of endothelial cell known in the art. Typically, the ECs are vascular endothelial cells. In some embodiments the ECs are primary endothelial cells. In some embodiments the ECs are mammalian ECs, such as human or non-human primate cells, or rabbit, rat, mouse, goat, pig, or other mammalian ECs. In some embodiments the ECs are primary human endothelial cells. ECs can be obtained from a variety of different tissues. In some embodiments the ECs are umbilical vein ECs (UVECs), such as human umbilical vein ECs (HUVECs). In some embodiments the ECs are nervous system ECs. In some embodiments the ECs are brain ECs. In some embodiments the ECs are spinal cord ECs. In some embodiments the ECs are olfactory bulb ECs. Other suitable ECs that can be used include those described previously as being suitable for E4ORF1-expression in U.S. Pat. No. 8,465,732, the contents of which are hereby incorporated by reference.

In some embodiments the ECs are autologous with respect to the subject into which they are to be transplanted/administered. In some embodiments the ECs are allogeneic with respect to the subject into which they are to be transplanted/administered. In some embodiments the ECs have the same MHC/HLA type as the subject into which they are to be transplanted/administered.

The E4ORF1+ ECs of the invention may exist in, or be provided in, various forms. For example, in some embodiments the ECs may comprise a population of ECs, such as an isolated population of ECs. In some embodiments the ECs may comprise a population of cells in vitro. In some embodiments the ECs may comprise a substantially pure population of cells. For example, in some embodiments at least about 50%, preferably at least about 75-80%, more preferably at least about 85-90%, and most preferably at least about 95% of the cells making up a total cell population will be E4ORF1+ ECs.

In some embodiments E4ORF1+ ECs may be provided in a composition (e.g. a therapeutic composition) that contains E4ORF1+ ECs and one or more additional cell types. In some embodiments such additional cell types are neural cell types, such as NPCs and/or glial cells.

In some embodiments the ECs are mitotically inactivated prior to use (e.g. therapeutic use) such that they cannot replicate. This can be achieved, for example, by using a chemical agent such as mitomycin C or by irradiating the engineered endothelial cells.

Methods of maintaining ECs in culture are known in the art and any suitable cell culture methods can be used. For example, E4ORF1+ ECs can be maintained in culture using methods known to be useful for maintaining other endothelial cells in culture, or, methods known to be useful for culturing E4ORF1+ ECs specifically, for example as described in U.S. Pat. No. 8,465,732, the contents of which are hereby incorporated by reference. In some embodiments E4ORF1+ ECs are maintained in culture in the absence of serum, or in the absence of exogenous growth factors, or in the absence of both serum and exogenous growth factors, or in the absence of exogenous angiogenic factors. E4ORF1+ ECs can also be cryopreserved. Various methods for cell culture and cell cryopreservation are known to those skilled in the art, such as the methods described in Culture of Animal Cells: A Manual of Basic Technique, 4th Edition (2000) by R. Ian Freshney (“Freshney”), the contents of which are hereby incorporated by reference.

### Neural Cells

In some embodiments the present invention involves neural cells, compositions that comprise neural cells, and methods of use of such neural cells and compositions.

As used herein the term “neural cells” encompasses neuronal cells and glial cells, and also neural stem cells (“NSCs”) and neural progenitor cells (“NPCs”). The terms “neural stem cells” and “neural progenitor cells” are used in accordance with their accepted meanings in the art. While stem cells and progenitor cells differ in their developmental potential (stem cells generally being at least pluripotent, while progenitor cells generally have a more limited developmental potential, i.e. multipotent at most), both NSCs and NPCs have the ability to produce both neuronal cells and glial cells. Some embodiments of the present invention involve NPCs that are neuronal progenitors and/or NPCs that are glial progenitors. Neuronal progenitors and glial progenitors have more limited potency than neural progenitors—with neuronal progenitors having the ability to produce neuron al cells and glial progenitors having the ability to produce glial cells.

In those embodiments of the present invention that involve neuronal cells, the neuronal cells may be any type of neuronal cell, including central and peripheral neurons. In some embodiments the neuronal cells are serotonergic neurons in particular. In some embodiments the neuronal cells are, or are derived from, primary neuronal cells. In other embodiments, the neuronal cells are derived from stem cells, progenitor cells, or non-neuronal cells. For example, in some embodiments the neuronal cells may be derived from neural stem cells, or neural progenitor cells, or neuronal progenitor cells. In some embodiments the neuronal cells may be derived from pluripotent stem cells, such as embryonic stem cells or induced pluripotent stem cells (iPSCs). Similarly, in some embodiments the neuronal cells may be derived by trans-differentiation from other differentiated cells such as differentiated non-neuronal cells.

In those embodiments of the present invention that involve glial cells, the glial cells may be, for example, astrocytes, oligodendrocytes, ependymal cells, radial glia, Schwann cells, satellite cells, enteric glial cells, or microglial cells. In some embodiments the glial cells are, or are derived from, primary glial cells. In other embodiments, the glial cells are derived from stem cells, progenitor cells, or non-glial cells. For example, in some embodiments the glial cells may be derived from neural stem cells, or neural progenitor cells, or glial progenitor cells. In some embodiments the glial cells may be derived from pluripotent stem cells, such as embryonic stem cells or induced pluripotent stem cells (iPSCs). Similarly, in some embodiments the glial cells may be derived by trans-differentiation from other differentiated cells such as differentiated non-glial cells.

In some embodiments the present invention involves compositions that comprise neural cells, and methods of use of such neural cells and compositions. The neural cells can be, or can be derived from, any type of neural cells known in the art. In some embodiments the neural cells are primary neural cells. In some embodiments the neural cells are mammalian neural cells, such as human or non-human primate cells, or rabbit, rat, mouse, goat, pig, or other mammalian neural cells. In some embodiments the neural cells are primary human neural cells. Neural cells can be obtained from a variety of different tissues. In some embodiments the neural cells are brain neural cells. In some embodiments the neural cells are spinal cord neural cells. In some embodiments the neural cells are olfactory bulb neural cells.

In some embodiments the neural cells are autologous with respect to the subject into which they are to be transplanted/administered. In some embodiments the neural cells are allogeneic with respect to the subject into which they are to be transplanted/administered. In some embodiments the neural cells have the same MHC/HLA type as the subject into which they are to be transplanted/administered.

The neural cells used in the compositions and methods of the present invention may exist in, or be provided in, various forms. For example, in some embodiments the neural cells may comprise a population of neural cells, such as an isolated population of neural cells. In some embodiments the neural cells may comprise a population of cells in vitro. In some embodiments the neural cells may comprise a substantially pure population of cells. For example, in some embodiments at least about 50%, preferably at least about 75-80%, more preferably at least about 85-90%, and most preferably at least about 95% of the cells making up a total cell population will be neural cells.

In some embodiments neural cells may be provided in a composition (e.g. a therapeutic composition) that contains neural cells and one or more additional cell types. In some embodiments such additional cell types are ECs, such as E4ORF1+ ECs.

In some embodiments, if the neural cells are mitotically active (such as NSCs and NPCs) they are mitotically inactivated prior to use (e.g. therapeutic use) such that they cannot replicate. This can be achieved, for example, by using a chemical agent such as mitomycin C, by irradiating the neural cells, or by exposing the cells to prolonged culturing conditions without supplements of mitogens such as basic fibroblast growth factor (bFGF).

Methods of maintaining neural cells in culture are known in the art and any suitable such method can be used in accordance with the present invention. Similarly, methods for cryopreserving neural cells are known in the art and can be used in accordance with the present invention. See, for example, Bonner J. F., Haas C. J., Fischer I. (2013) “Preparation of Neural Stem Cells and Progenitors: Neuronal Production and Grafting Applications.” In: Amini S., White M. (eds) Neuronal Cell Culture. Methods in Molecular Biology (Methods and Protocols), Vol 1078. Humana Press, Totowa, N.J.

Compositions Comprising Endothelial Cells and/or Neural Cells

In some embodiments the E4ORF1+ ECs and/or neural cells may be provided in the form of a composition containing the specified cells and one or more additional components and/or additional cell types. For example, in some embodiments a composition comprising the recited cells together in a carrier solution is used. Such carrier solutions may consist of, or contain, for example, a physiological saline solution, a cell suspension medium, a cell culture medium, or the like. In some embodiments a composition comprising the recited cells together with a biocompatible matrix material may be used. In some embodiments, the biocompatible matrix material is one that is solid at room temperature. In some embodiments, the biocompatible matrix material is one that is liquid at room temperature. In some embodiments, the biocompatible matrix material is one that is solid at body temperature (i.e. around 37° C.). In some embodiments, the biocompatible matrix material is one that is liquid at body temperature (i.e. around 37° C.). In some embodiments, the biocompatible matrix material is one that is solid when on ice and/or when refrigerated (i.e. from around 0° C. to around 4° C.). In some embodiments, the biocompatible matrix material is one that is liquid when on ice and/or when refrigerated (i.e. from around 0° C. to around 4° C.). In some embodiments, the biocompatible matrix material is one that is liquid at room temperature and remains in liquid during process of administration to a subject according to the methods of the present invention.

In certain embodiments the biocompatible matrix material comprises, consists of, or consists essentially of, de-cellularized animal tissue, or one or more extracellular matrix (“ECM”) components such as collagen, laminin, and/or fibrin. In some embodiments the biocompatible scaffold comprises at least about 5%, 10%, 20%, 30%, 40%, 50%, 60%, 70%, 80%, 90% or 95% collagen. In some embodiments the biocompatible scaffold comprises at least about 5%, 10%, 20%, 30%, 40%, 50%, 60%, 70%, 80%, 90% or 95% laminin. In some embodiments the biocompatible scaffold comprises at least about 5%, 10%, 20%, 30%, 40%, 50%, 60%, 70%, 80%, 90% or 95% fibrin. In some embodiments the biocompatible scaffold does not comprise hyaluronic acid. In some embodiments the biocompatible scaffold does not comprise more than about 5%, 4%, 3%, 2%, 1%, or 0.5% hyaluronic acid. In some embodiments the biocompatible comprises Matrigel. In some embodiments the biocompatible scaffold does not comprise Matrigel. In some embodiments the biocompatible scaffold material may be selected depending on the tissue location into which it is to be implanted, for example based on its biomechanical properties or any other biological properties.

In some embodiments each of the compositions recited herein (e.g. those consisting of or containing carrier solutions and/or biocompatible matrix materials) may be “therapeutic compositions”—meaning that the components of the composition are suitable for administration to a subject, such as a human subject. Other therapeutically acceptable agents can be included if desired. One of ordinary skill in the art can readily select suitable agents to be included in the therapeutic compositions depending on the intended use.

In some embodiments E4ORF1+ ECs and neural cells may be provided together in the same composition—i.e. as a mixture of cell types. In some of such embodiments the ratio of E4ORF1+ ECs to neural cells may be about 1:10, or about 1:9, or about 1:8, or about 1:7, or about 1:6, or about 1:5, or about 1:4, or about 1:3, or about 1:2, or about 1:1, or about 2:1, or about 3:1, or about 4:1, or about 5:1, or about 6:1, or about 7:1, or about 8:1, or about 9:1, or about 10:1.

### Therapeutic Methods

In some embodiments the present invention provides methods of treating SCI in subjects in need thereof. Such methods involve transplanting/administering E4ORF1+ ECs and neural cells to the site of a SCI in a subject. In some embodiments the E4ORF1+ ECs and the neural cells are administered concurrently. In some embodiments the E4ORF1+ ECs and the neural cells are administered at different times. In some embodiments the E4ORF1+ ECs and the neural cells are administered together in a composition that comprises both cell types. In some embodiments the E4ORF1+ ECs and the neural cells are administered separately in two separate compositions—one of which comprises the E4ORF1+ ECs and the other or which comprises the neural cells.

In some embodiments the ratio of E4ORF1+ ECs to neural cells that is transplanted/administered to the subject is about 1:10, or about 1:9, or about 1:8, or about 1:7, or about 1:6, or about 1:5, or about 1:4, or about 1:3, or about 1:2, or about 1:1, or about 2:1, or about 3:1, or about 4:1, or about 5:1, or about 6:1, or about 7:1, or about 8:1, or about 9:1, or about 10:1.

In some embodiments the number of E4ORF1+ ECs that is transplanted/administered to the subject is about 100,000 cells, or about 250,000, cells or about 500,000 cells, or about 1,000,000 cells, or about 1,500,000 cells, or about 2,000,000 cells, or about 3,000,000 cells, or about 4,000,000 cells, or about 5,000,000 cells, or about 6,000,000 cells, or about 7,000,000 cells, or about 8,000,000 cells, or about 9,000,000 cells, or about 10,000,000 cells.

In some embodiments the E4ORF1+ ECs and/or the neural cells are administered to the site of the SCI by injection, by infusion, by surgical implantation, or by some other suitable form of delivery of the cell. For example, in some embodiments the E4ORF1+ ECs and/or the neural cells are administered to the site of the SCI by injection or infusion of a liquid composition comprising the cells. Similarly, in other embodiments the E4ORF1+ ECs and/or the neural cells are administered to the site of the SCI by surgical implantation of the cells in a solid matrix. Any suitable technique known in the art for administration of cells to the spinal cord or to a spinal cord lesion can be used. The precise details of the technique used to transplant/administer the cells to the site of a SCI can be determined taking into account the specific circumstances including, but not limited to, the species of the subject, the age of the subject, the location of the SCI, etc. Typically, the details of the technique used to transplant/administer the cells to the site of the SCI will be determined by a physician, such as the surgeon or other practitioner performing the transplantation/administration procedure, and/or with advice from a scientific advisory board.

The timing of the administration of the E4ORF1+ ECs and/or the neural cells to the subject can be any suitable time after the creation of the injury. In the case of human subjects, a physician will typically make a determination about the timing of the administration. In some embodiments, the E4ORF1+ ECs and/or the neural cells are administered to the subject within the acute phase after the creation of the SCI injury. In the case of human subjects, the acute phase is typically considered to be within about 0-2 days following the creation of the SCI injury. In some embodiments, the E4ORF1+ ECs and/or the neural cells are administered to the subject within the subacute phase after the creation of the SCI injury. In the case of human subjects, the subacute phase is typically considered to be within about 3-14 days following the creation of the SCI injury. In some embodiments, the E4ORF1+ ECs and/or the neural cells are administered to the subject within the intermediate phase after the creation of the SCI injury. In the case of human subjects, the intermediate phase is typically considered to be within about 2 weeks to 6-months following the creation of the SCI injury. In some embodiments, the E4ORF1+ ECs and/or the neural cells are administered to the subject within the chronic phase after the creation of the SCI injury. In the case of human subjects, the intermediate phase is typically considered to be more than 6-months following the creation of the SCI injury. In some embodiments, the E4ORF1+ ECs and/or the neural cells are administered to the subject within about 1 week of the creation of the SCI injury. In some embodiments, the E4ORF1+ ECs and/or the neural cells are administered to the subject within about 2 week2 of the creation of the SCI injury. In some embodiments, the E4ORF1+ ECs and/or the neural cells are administered to the subject within about 3 weeks of the creation of the SCI injury. In some embodiments, the E4ORF1+ ECs and/or the neural cells are administered to the subject within about 4 weeks of the creation of the SCI injury.

### Model Systems

In addition to being useful for therapeutic applications, the transplantation methods of the present invention may also be useful in a variety of other contexts, for example in the production of model systems useful in studying SCI and possible treatments for SCI, including drug screening methods. For example, in some embodiments the present invention provides methods for assessing the effect of one or more candidate agents or candidate cell types on SCI or SCI repair comprising performing a treatment method as described herein and testing the effects or one or more candidate agents or candidate cell types thereon.

### Kits

The present invention also provides kits for carrying out the various methods described herein. Such kits may contain any of the components described herein, including, but not limited to, E4ORF1 sequences (for example in a vector), endothelial cells, E4ORF1+ endothelial cells, neural cells (such as neurons, glia, NSCs, NPCs, neuronal progenitors, or glial progenitors), means or compositions for detection of E4ORF1 sequences or E4ORF1 polypeptides (e.g. nucleic acid probes, antibodies, etc.), media or compositions useful for maintaining or expanding E4ORF1+ neural cells or neural cells, means or compositions for administering E4ORF1+ ECs and/or neural cells to subjects, instructions for use, containers, culture vessels, and the like, or any combinations thereof.

Certain aspects of the present invention may be further described in the following non-limiting Examples.

### EXAMPLES

### Example 1

### Materials & Methods

### Summary of Materials & Methods

Animal Model: Adult female Sprague-Dawley rats. Injury Model: Mid-cervical (C3-4) lateralized contusion. Treatment: Injection of ECs alone, or in combination with NPCs. Treatment time: Single delivery 1-week post-injury. Treatment dose: 10 microliters of cells at 100,000 cells/ul in culture media (HBSS). Route of administration: Direct injection into lesion site. Method for delivery: Injection via surgical syringe (Hamilton) with a 30-gauge steel needle. Experimental time-course: 7 weeks from day of injury. Anatomical outcome measures: neuroanatomical tracing & immunohistochemistry (observe effects on cavitation, vascularity, and axonal growth. Functional outcome measures: terminal electrophysiology (observe effects on muscle activity). Behavioral outcome measures: weekly plethysmography assessment (observe effects on breathing patterns (frequency of breathing, tidal volume, minute ventilation).

### Detailed Materials & Methods

Neural Progenitor Cell Isolation & Culture: The detailed Neural Progenitor Cell (NPCs) isolation protocol used in these studies can be found in Bonner et al. (2013) (Bonner J. F., Haas C. J., Fischer I. (203) Preparation of Neural Stem Cells and Progenitors: Neuronal Production and Grafting Applications. In: Amini S. White M. (eds) Neuronal Cell Culture. Methods in Molecular Biology (Methods and Protocols), vol 1078. Humana Press, Totowa, N.J.), The NPCs are isolated from E13.5-14 rat (Fischer 344-Tg UBC-eGFP) spinal cords, or from E12.5-13 mouse spinal cord. Dissected spinal cord tissue is mechanically and enzymatically (Trypsin, Life Technologies #25200-056) dissociated and cultured for 3 days in Culture Media prior to cryoprotection in Freezing Media (ThermoFischer #12648010) and storage in liquid nitrogen until needed. Cells are thawed one day prior to combination with ECs by seeding 3×10⁶ or 6×10⁶ NPCs onto poly-L-lysine (Sigma-Aldrich, #P8920) and laminin (ThermoFischer, #23017015) coated T75 flasks and cultured in Culture Media. The components of the Culture Media are as follows: DMEM/F12 containing 25 mg/mL bovine serum albumin, B-27 supplement (Life Technologies, #17504-044), N2 supplement (Life Technologies, #17502-048), 10 ng/mL basic fibroblast growth factor (bFGF; Peprotech, #450-10, Rocky Hill, N.J.), and 20 ng/mL neurotrophin-3 (NT-3; Peprotech, #450-03).

Spinal Endothelial Cell Isolation & Culture: Spinal cords and olfactory bulbs are dissected from 3-4 week old Sprague Dawley rats and immediately placed in dissection buffer (L15 medium supplemented with 1XB27; L15: #11415064, B27: #17504044, ThermoFischer). The tissue is dissociated using a combination of mechanical and enzymatic dissociation/digestion methods. The completely digested tissue is spun down in a centrifuge (×400 g, 5 minutes) and the pellet is resuspended in EC culture media and cultured for 2 days. Lentiviral particles encoding E4ORF1 are added into the media. Fresh EC media is added into the cultures every 3 days. The ECs are cultured to reach at least 80% confluency prior to cryopreservation.

SCI Model: Mid-cervical (C3-4) spinal cord contusion injury is modeled in the adult female rat using the Infinite Horizon pneumatic impactor (preset impact force of 200 kilodynes, 0 second dwell time). This injury compromises the phrenic motor circuitry and impairs diaphragm function, which will be assessed using bilateral terminal muscle electromyography (EMG). This injury also results in a loss of about 50% of the spinal motoneurons that comprise the phrenic motor pool (innervating the diaphragm), and denervates phrenic motoneurons caudal to injury. This anatomical deficit results in attenuated muscle function ipsilateral to injury, and an impaired response to increased respiratory drive (or respiratory insufficiency). Increased respiratory drive is stimulated by exposing animals to either hypoxic (10% inhaled 02) or hypercapnic (7% inhaled C02) gases. While some spontaneous functional plasticity can occur in this model, the extent of attributable recovery is limited, and deficits persist. Figure A provides a schematic representation of this SCI model.

Treatment: Donor cells are injected directly into the lesion site (single route of administration) 1-week post-injury, at a dose of 1 million cells. This delayed (sub-acute) treatment time is comparable to that currently used for other cell therapy studies. In animals treated sub-acutely, the spinal cord is surgically re-exposed 1-week post-injury and a small dural incision made immediately overlying the injury. Cells suspended in Hanks Balanced Salt Solution (HBSS) are drawn up into a glass syringe with a 30-gauge custom (30 degree angle) needle attached (World Precision Instruments) The syringe is placed into a micromanipulator and positioned over the exposed spinal cord. The needle tip is inserted intra-spinally to reach the lesion epicenter. After delivery, the needle is withdrawn, the animal sutured and given post-operative medication, and allowed to recover in a clean environment with close monitoring.

Functional Outcome Measures: Ventilatory function (tidal volume, breathing frequency and minute ventilation) is assessed using whole-body plethysmography, prior to and weekly following injury in animals from all treatment groups. Ventilatory data collected from uninjured animals can be used for comparison with the treatment groups. Terminal diaphragm electromyography (EMG) is used to determine whether treatment promotes phrenic motor recovery. Terminal phrenic neurograms or weekly diaphragm EMG in awake animals (using telemeters) can also be used during plethysmographic assessment.

Anatomical Outcome Measures: Retrograde tracing methods are employed to map the phrenic motor circuitry, as has been done previously^{14,19,23}. Three days prior to the end of the experiment (6.5 weeks post-injury), animals undergo surgery to expose the diaphragm and pseudorabies virus (PRV) is delivered to the hemidiaphragm ipsilateral to injury, as previously described^{14,23}. This anatomical tracing approach enables characterization of the number of interneurons synaptically-integrated with the phrenic motoneurons. The number of motor- and interneurons are quantified and compared with previously obtained data from uninjured animals. The number of cells within the raphe and reticular nuclei are quantified from animals traced with PRV. PRV labeled interneurons rostral and caudal to the injury are quantified according to their laminae distribution. Sections from labeled tissue are analyzed to determine density of labeled axons and number of PRV infected neurons.

This technique can also reveal the number of donor neurons that become synaptically integrated with the injured host spinal cord. (Some donor NPCs may differentiate into mature neurons and synaptically integrate with the injured phrenic motor circuitry.)

Immunohistochemistry is used to identify the number of serotonergic axons detectable and differences between groups are assessed. Additional axon populations can also be assessed, for example using anterograde tracing methods. Anterograde tracing methods include, but are not limited to, delivery of biotinylated dextran amine (BDA) to spontaneously active cells within the ventral respiratory column via iontophoresis, injection of BDA or other anterograde tracer to raphe or other brainstem nuclei, injection of BDA or other anterograde tracer to the motor, sensory or other cortex.

Additional immunohistochemistry with anti-endothelial cell antibody (RECA) or other primary antibodies is used to assess the extent of vascularity surrounding and potentially within the lesion epicenter. To test whether newly formed vessels are functioning, immunohistochemistry for plasma proteins (to determine vessel content and test for any unwanted passage of proteins into the nervous system) is performed.

### Example 2

### Effect of Transplantation of NPCs and E4ORF1+ ECs in Spinal Cord Injury Model

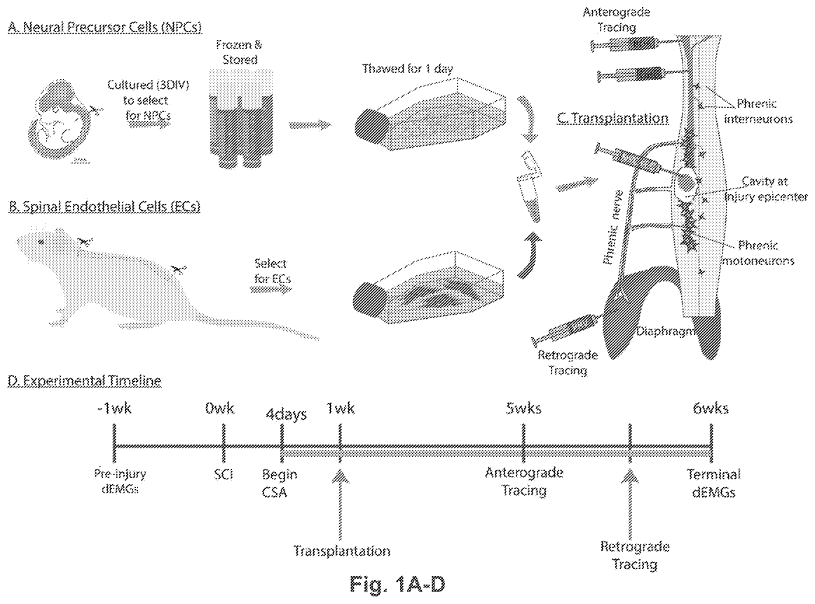

FIG. 1. provides a schematic diagram of the methods and timeline employed in the described experiments. FIG. 1A. Neural progenitor cells (NPCs) were isolated from developing rat spinal cord, cultured, frozen, and thawed 1 day prior to transplantation. FIG. 1B. E4ORF1 expressing mouse spinal endothelial cells (ECs) were thawed and cultured with lenti-GFP virus prior to transplantation. FIG. 1C. NPCs and ECs were combined at a 1:1 ratio (1,000,000 cells total) and transplanted into the lesion epicenter, 1 week after contusion spinal cord injury. A battery of anatomical (anterograde, and retrograde tracers) and functional (terminal diaphragm electromyography, dEMGs) assessments were used to evaluate the efficacy of this transplant paradigm. The experimental timeline is shown in FIG. 1D.

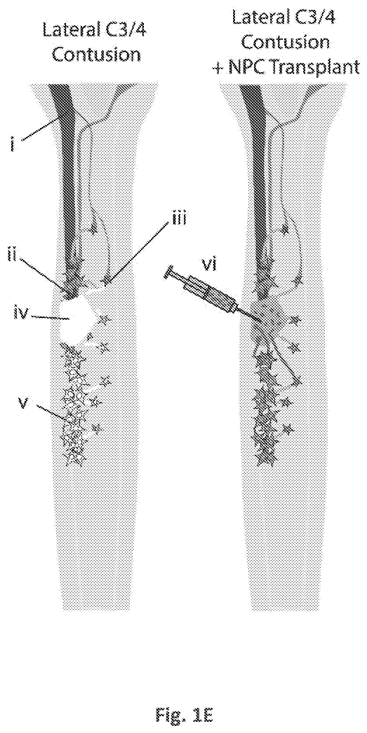

Phenotypic analysis of transplanted NPCs and ECs reveals differentiation into GFAP positive glia 6 weeks after transplantation. As shown in FIG. 2A transplanted, GFP-expressing NPCs and ECs (FIG. 2A) result in high expression of GFAP positive glia (FIG. 2B) 6 weeks after transplantation. FIG. 2C shows a scatter plot used for calculating the Manders colocalization coefficient, where Quadrant 1 (Q1) represents pixels that have high GFAP intensities and low GFP intensities; Q2 represents pixels with high intensity levels in both GFAP and GFP channels and Q4 represents high GFP and low GFAP intensities. Q3 represents pixels that have low intensity levels in both channels. This assessment revealed an average Manders coefficient of 0.96 (N=3), see Table 1.

TABLE 1
Manders colocalization coefficient
for Transplanted NPCs and ECs.

| Animal # | Pearson | Manders |
|---|---|---|
| 1 | 0.863 | 0.976 |
| 2 | 0.694 | 0.959 |
| 3 | 0.585 | 0.940 |
| Average | 0.714 | 0.958 |
| STD | 0.140 | 0.018 |
| SEM | 0.081 | 0.011 |

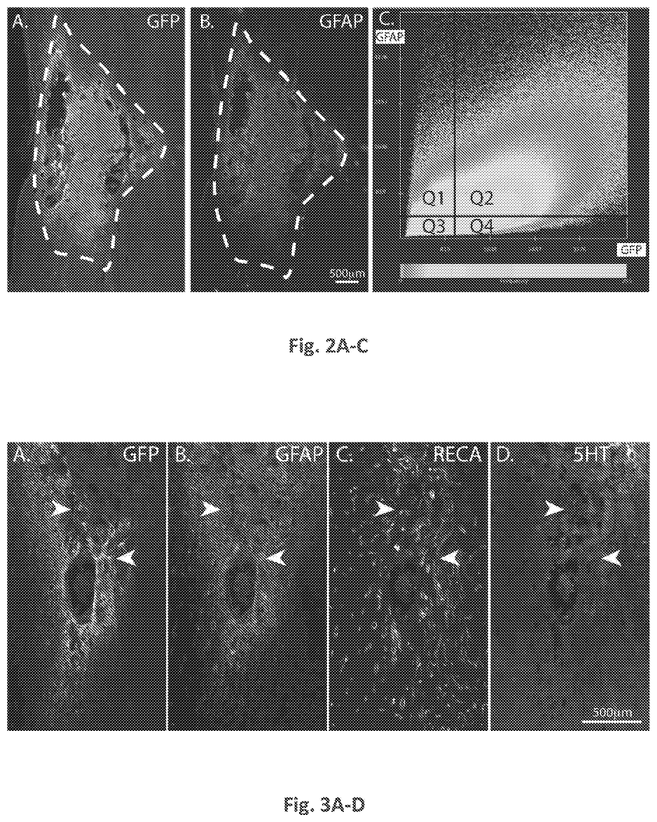

Transplantation of NPCs with Endothelial Cells (ECs) results in enhanced serotonergic growth through the lesion cavity. Transplanted GFP labeled NPCs and ECs survive 6 weeks after transplantation (FIG. 3A), yield GFAP positive glia (FIG. 3B) and result in increased vascularization throughout the lesion cavity as depicted by Rat Endothelial Cell Antigen (RECA) staining (FIG. 3C). The combinatorial transplant (NPCs+ECs) results in host serotonergic (5HT) growth through the lesion cavity (FIG. 3D). In FIG. 3A-D the white arrows show growing axons. Scale bars are as indicated.

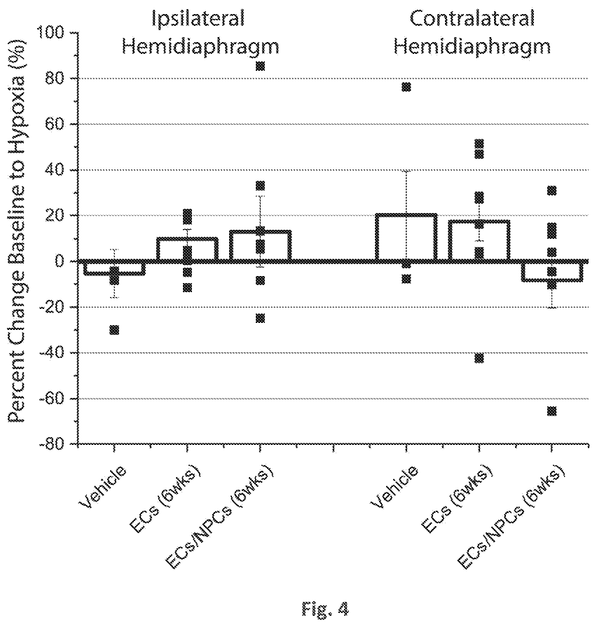

Transplantation of Neural Progenitor Cells (NPCs) with Endothelial Cells (ECs) results in modest diaphragm recovery 6 weeks after transplantation. FIG. 4. Diaphragm function was assessed 6 weeks after transplantation using terminal diaphragm electromyography (dEMGs) during baseline (normal breathing) and under a respiratory challenge (hypoxia, 10% 02). The percent change (i.e. the animal's ability to respond to the respiratory challenge) is represented in FIG. 4 with each dot an average of 40 second recording from each animal. The bar graphs represent the average of each indicated group.

### Example 3

### Effect of Transplantation of Glial Progenitors or Glial Cells Together with E4ORF1+ ECs in Spinal Cord Injury Model

As described above in Example 2, it was found that, following NPC transplantation, the NPCs differentiated into GFAP positive glia about 6 weeks after transplantation. As such, we hypothesized that the SCI repair described above may also be achieved if glial progenitors or glia (instead of NPCs) are transplanted together with E4ORF1+ ECs.

To test this hypothesis the following experiments are performed, with all methods being as described above unless stated otherwise.

Glial progenitors and/or glia are obtained. Spinal endothelial cells (ECs) are obtained and transduced to produce E4ORF1+ ECs as described above. A first combination of glial progenitors and E4ORF1+ ECs and a second combination of glia and E4ORF1+ ECs are transplanted into the lesion epicenter of the SCI model described above. A battery of anatomical (anterograde, and retrograde tracers) and functional (terminal diaphragm electromyography, dEMGs; telemetric chronically implanted diaphragm electromyography in awake animals) assessments are used to evaluate the efficacy of these transplant paradigms.

### Example 4

### Exemplary Human Clinical Trial

E4ORF1+ ECs and NPCs are administered to a human subject having a SCI by direct local injection into the injury site during the subacute phase after the event that created the injury. Approximately 1,000,000 cells in total (at a 1:1 ratio of E4ORF1+ ECs to NPCs) are administered in a composition comprising a physiological saline. Following the procedure, the treatment outcome is assessed by monitoring one or more well-known parameters indicative of either anatomical recovery at the injury site (for example using suitable tracers and imaging methodologies) or functional recovery (for example electrophysiological measures and/or assessment of motor and/or sensory function). The treatment parameters can be adjusted in different subjects and the effect of these adjustments on the treatment outcome can be measured. The treatment parameters that can be adjusted include, but are not limited to, the total cell number administered, the ratio of E4ORF1+ ECs to NPCs, the constituents of the composition (e.g. buffers, excipients, growth factors, biocompatible matrices), the administration method (e.g. injection vs. infusion), the administration location, the administration timing relative to that of the event that created the injury (e.g. during the acute vs. subacute phase, or within less than 1 week, about 1 week, about 2 weeks, or more than 2 weeks following the injury, etc.), the source of the E4ORF1+ ECs and the source of the NPCs.

### REFERENCE LIST

1. Lane, M. A., Lepore, A. C. & Fischer, I. Improving the therapeutic efficacy of neural progenitor cell transplantation following spinal cord injury. Expert Rev Neurother, 1-8 (2016).
2. Rauch, M. F., et al. Engineering angiogenesis following spinal cord injury: a coculture of neural progenitor and endothelial cells in a degradable polymer implant leads to an increase in vessel density and formation of the blood-spinal cord barrier. The European journal of neuroscience 29, 132-145 (2009).
3. Nolan, D. J., et al. Molecular signatures of tissue-specific microvascular endothelial cell heterogeneity in organ maintenance and regeneration. Dev Cell 26, 204-219 (2013).
4. Rauch, M. F., Michaud, M., Xu, H., Madri, J. A. & Lavik, E. B. Co-culture of primary neural progenitor and endothelial cells in a macroporous gel promotes stable vascular networks in vivo. J Biomat Sci-Polym E 19, 1469-1485 (2008).
5. Ford, M. C., et al. A macroporous hydrogel for the coculture of neural progenitor and endothelial cells to form functional vascular networks in vivo. Proceedings of the National Academy of Sciences of the United States of America 103, 2512-2517 (2006).
6. Jin, Y., et al. Transplantation of human glial restricted progenitors and derived astrocytes into a contusion model of spinal cord injury. J Neurotrauma 28, 579-594 (2011).
7. Lepore, A. C., et al. Human glial-restricted progenitor transplantation into cervical spinal cord of the SOD1 mouse model of ALS. PLoS One 6, e25968 (2011).
8. Hormigo, K. M., et al. Enhancing neural activity to drive respiratory plasticity following cervical spinal cord injury. Exp Neurol 287, 276-287 (2017).
9. Nair, J., et al. Histological identification of phrenic afferent projections to the spinal cord. Respir Physiol Neurobiol 236, 57-68 (2017).
10. Vinit, S., et al. Interdisciplinary approaches of transcranial magnetic stimulation applied to a respiratory neuronal circuitry model. PLoS One 9, e113251 (2014).
11. Lee, K. Z., et al. Intraspinal transplantation and modulation of donor neuron electrophysiological activity. Exp Neurol 251, 47-57 (2014).
12. Hoh, D. J., Mercier, L. M., Hussey, S. P. & Lane, M. A. Respiration following spinal cord injury: evidence for human neuroplasticity. Respir Physiol Neurobiol 189, 450-464(2013).
13. Dougherty, B. J., Lee, K. Z., Lane, M. A., Reier, P. J. & Fuller, D. D. Contribution of the spontaneous crossed-phrenic phenomenon to inspiratory tidal volume in spontaneously breathing rats. J Appl Physiol (1985) 112, 96-105 (2012).
14. Lane, M. A., et al. Respiratory function following bilateral mid-cervical contusion injury in the adult rat. Exp Neurol 235, 197-210 (2012).
15. Dougherty, B. J., et al. Recovery of inspiratory intercostal muscle activity following high cervical hemisection. Respir Physiol Neurobiol 183, 186-192 (2012).
16. Lane, M. A. Spinal respiratory motoneurons and interneurons. Respir Physiol Neurobiol 179, 3-13 (2011).
17. Qiu, K., Lane, M. A., Lee, K. Z., Reier, P. J. & Fuller, D. D. The phrenic motor nucleus in the adult mouse. Exp Neurol 226, 254-258 (2010).
18. White, T. E., et al. Neuronal progenitor transplantation and respiratory outcomes following upper cervical spinal cord injury in adult rats. Exp Neurol 225, 231-236 (2010).
19. Lane, M. A., Lee, K. Z., Fuller, D. D. & Reier, P. J. Spinal circuitry and respiratory recovery following spinal cord injury. Respir Physiol Neurobiol 169, 123-132 (2009).
20. Sandhu, M. S., et al. Respiratory recovery following high cervical hemisection. Respir Physiol Neurobiol 169, 94-101 (2009).
21. Fuller, D. D., et al. Graded unilateral cervical spinal cord injury and respiratory motor recovery. Respir Physiol Neurobiol 165, 245-253 (2009).
22. Lane, M. A., Fuller, D. D., White, T. E. & Reier, P. J. Respiratory neuroplasticity and cervical spinal cord injury: translational perspectives. Trends Neurosci 31, 538-547 (2008).
23. Lane, M. A., et al. Cervical prephrenic interneurons in the normal and lesioned spinal cord of the adult rat. The Journal of comparative neurology 511, 692-709 (2008).
24. Zholudeva, L., et al. Excitatory Neural Precursor Cells Promote Respiratory Recovery after a Cervical Spinal Cord Injury. J Neurotraum 33, A137-A137 (2016).
25. Spruance, V., Zholudeva, L., Negron, K., Bezdudnaya, T. & Lane, M. Short and Long Term Effects of Neural Progenitor Transplantation to Promote Recovery of Breathing after Spinal Cord Injury. J Neurotraum 33, A73-A74 (2016).
26. Zholudeva, L. V., et al. Axonal Outgrowth of Neural Precursor Cells Transplanted Into the Contused Cervical Spinal Cord. Cell Transplantation 25, 779-779 (2016).
27. Spruance, V. M., et al. Transplantation of Progenitor Cells Promotes Respiratory Recovery After Spinal Cord Injury. Cell Transplantation 25, 773-773 (2016).
28. Spruance, V., et al. Transplantation of neural progenitor cells promotes respiratory recovery after cervical spinal cord injury. Journal of Neurochemistry 134, 370-370 (2015).
29. Spruance, V. M., et al. Improving Respiratory Function With the Transplantation of Neural Progenitors Following Injury. Cell Transplantation 23, 783-783 (2014).
30. Lopez, C., et al. Synaptic integration of transplanted cells with phrenic circuitry following high cervical spinal cord injury in adult rat. in International Symposium on Neural Regeneration, Vol. International Symposium on Neural Regeneration (Asilomar, C A, 2011).
31. Sanchez, D. E., et al. Intraspinal grafts of neural progenitors improves respiratory function following mid-cervical contusion injury in adult rats. in International Symposium on Neural Regeneration, Vol. International Symposium on Neural Regeneration—Poster Award Winner (Asilomar, C A, 2011).
32. Goshgarian, H. G. The crossed phrenic phenomenon and recovery of function following spinal cord injury. Respir Physiol Neurobiol 169, 85-93 (2009).
33. Fuller, D. D., et al. Modest spontaneous recovery of ventilation following chronic high cervical hemisection in rats. Exp Neurol 211, 97-106 (2008).
34. Herrera et al. Sustained expression of vascular endothelial growth factor and angiopoietin-1 improves blood-spinal cord barrier integrity and functional recovery after spinal cord injury. Neurotrauma. 2010 November; 27(11):2067-76.
   The present invention is further described by the following claims.

We claim:

1. A method of treating spinal cord injury (SCI) in a mammalian subject in need thereof, the method comprising administering an effective amount of E4ORF1+ CNS-derived endothelial cells (E4ORF1+ ECs) and an effective amount of neural progenitor cells (NPCs) to a subject having an SCI locally at the site of the SCI, thereby treating the SCI in the subject, wherein the treatment results in the growth and/or extension of functional axons through the site of the spinal cord injury and a detectable improvement in an SCI-associated sensory or motor deficit.

2. The method of claim 1, wherein the ratio of the E4ORF1+ ECs to NPCs neural cells is about 1:1.

3. The method of claim 1 wherein the E4ORF1+ ECs and the NPCs are administered to the subject in a physiological saline solution.

4. The method of claim 1 wherein the E4ORF1+ ECs and the NPCs are administered to the subject together.

5. The method of claim 1 wherein the E4ORF1+ ECs and the NPCs are administered to the subject separately.

6. The method of claim 1 wherein the E4ORF1+ ECs and the NPCs are administered to the subject during the subacute phase of the SCI injury.

7. The method of claim 1 wherein the E4ORF1+ ECs and the NPCs are administered by direct injection at the site of the SCI.

8. The method of claim 1, wherein the E4ORF1+ ECs and/or the NPCs are administered to the subject in a biocompatible matrix material.

9. The method of claim 1, wherein the E4ORF1+ ECs and/or the NPCs are administered to the subject in a solid 3D biocompatible matrix material.

10. The method of claim 1, wherein the E4ORF1+ ECs and/or the NPCs are not administered to the subject in a biocompatible matrix material.

11. A composition for comprising an effective amount of E4ORF1+ CNS-derived endothelial cells (E4ORF1+ ECs) and an effective amount of neural progenitor cells (NPCs) for use in a method according to claim 1.

12. The composition of claim 11, wherein the ratio of the E4ORF1+ ECs to NPCs neural cells is about 1:1.

13. The composition of claim 11, wherein the composition comprises physiological saline.

14. The composition of claim 11, wherein the composition comprises a biocompatible matrix material.

15. The composition of claim 11, wherein the composition does not comprise a biocompatible matrix material.

16. A method of treating spinal cord injury (SCI) in a subject in need thereof, the method comprising: administering: (a) E4ORF1+ endothelial cells (ECs), and (b) neural cells, to a subject having a SCI, wherein the E4ORF1+ ECs and the neural cells are administered locally at the site of the SCI, thereby treating the SCI in the subject.

17. The method of claim 16, wherein the ECs are vascular ECs.

18. The method of claim 16, wherein the ECs are primary ECs.

19. The method of claim 16, wherein the ECs are cultured EC cells from an EC cell line.

20. The method of any claim 16, wherein the ECs are mammalian ECs.

21. The method of claim 16, wherein the ECs are primate ECs.

22. The method of any of claims 16-21, wherein the ECs are human ECs.

23. The method of claim 22, wherein the ECs are rabbit, rat, mouse, guinea pig, goat, pig, sheep, cow, horse, cat or dog mammalian ECs.

24. The method of any of claims 16-23, wherein the ECs are selected from the group consisting of umbilical vein ECs (UVECs), brain ECs, spinal cord ECs, or olfactory bulb ECs.

25. The method of any of claims 16-24, wherein the ECs are allogeneic with respect to the subject.

26. The method of any of claims 16-24, wherein the ECs are autologous with respect to the subject.

27. The method of any of claims 16-24, wherein the ECs have the same MHC/LA type as the subject.

28. The method of any of claims 16-27, wherein the ECs are mitotically inactive.

29. The method of any of claims 16-27, wherein the ECs are differentiated ECs.

30. The method of any of claims 16-27, wherein the ECs are adult ECs.

31. The method of any of claims 16-30, wherein the neural cells are primary neural cells.

32. The method of any of claims 16-30, wherein the neural cells are cells from a cultured neural cell line.

33. The method of any of claims 16-32, wherein the neural cells are mammalian neural cells.

34. The method of any of claims 16-33, wherein the neural cells are primate neural cells.

35. The method of any of claims 16-34, wherein the neural cells are human neural cells.

36. The method of claim 33, wherein the neural cells are rabbit, rat, mouse, guinea pig, goat, pig, sheep, cow, horse, cat or dog mammalian neural cells.

37. The method of any of claims 16-36, wherein the neural cells are selected from the group consisting of neuronal cells, glial cells, neural stem cells, neural progenitor cells, neuronal progenitor cells, glial progenitor cells.

38. The method of any of claims 16-36, wherein the neural cells are allogeneic with respect to the subject.

39. The method of any of claims 16-36, wherein the neural cells are autologous with respect to the subject.

40. The method of any of claims 16-36, wherein the neural cells have the same MHC/HLA type as the subject.

41. The method of any of 16-40 claims, wherein the neural cells are mitotically inactive.

42. The method of any of claims 16-41, wherein the neural cells are differentiated neural cells.

43. The method of any of claims 16-42, wherein the neural cells are adult neural cells.

44. The method of any of claims 16-43, wherein the subject is a mammal.

45. The method of any of claims 16-44, wherein the subject is a primate.

46. The method of any of claims 16-45, wherein the subject is a human.

47. The method of claim 44 wherein the subject is a rabbit, rat, mouse, guinea pig, goat, pig, sheep, cow, horse, cat or dog.

48. The method of any of the preceding claims, wherein the E4ORF1+ ECs comprise a nucleic acid molecule that encodes an adenovirus E4ORF1 polypeptide.

49. The method of claim 48, wherein the nucleic acid molecule is in a vector.

50. The method of claim 49, wherein the vector is a retroviral vector.

51. The method of claim 50, wherein the retroviral vector is a lentiviral vector.

52. The method of claim 50, wherein the retroviral vector is a Maloney murine leukemia virus (MMLV) vector.

53. The method of any of claims 48-52, wherein the nucleic acid molecule is integrated into the genomic DNA of the ECs.

54. The method of any of claims 16-53, wherein the ratio of the E4ORF1+ ECs to neural cells is about 1:10, or about 1:9, or about 1:8, or about 1:7, or about 1:6, or about 1:5, or about 1:4, or about 1:3, or about 1:2, or about 1:1, or about 2:1, or about 3:1, or about 4:1, or about 5:1, or about 6:1, or about 7:1, or about 8:1, or about 9:1, or about 10:1.

55. The method of any of claims 16-54, wherein either: (a) the E4ORF1+ ECs, (b) the neural cells, or (c) both the E4ORF1+ ECs and the neural cells, are administered to the subject in a physiological saline solution.

56. The method of any of claims 16-54, wherein either: (a) the E4ORF1+ ECs, (b) the neural cells, or (c) both the E4ORF1+ ECs and the neural cells, are administered to the subject in a biocompatible matrix material.

57. The method of any of claims 16-56, wherein the E4ORF1+ ECs and the neural cells are administered to the subject concurrently.

58. A composition comprising E4ORF1+ ECs and neural cells.

59. A composition comprising E4ORF1+ ECs and neural cells for use in a method of treating SCI in a subject in need thereof.

60. A composition comprising E4ORF1+ ECs and neural cells for use in a method of treating spinal cord injury (SCI) according to any one of claims 16-57.

61. The composition of claim 58, 59, or 60, wherein the ECs are vascular ECs.

62. The composition of any of claims 58-61, wherein the ECs are primary ECs.

63. The composition of any of claims 58-61, wherein the ECs are cells from a cultured EC cell line.

64. The composition of any of claims 58-63, wherein the ECs are mammalian ECs.

65. The composition of any of claims 58-64, wherein the ECs are primate ECs.

66. The composition of any of claims 58-65, wherein the ECs are human ECs.

67. The composition of claim 64, wherein the ECs are rabbit, rat, mouse, guinea pig, goat, pig, sheep, cow, horse, cat or dog mammalian ECs.

68. The composition of any of claims 58-67, wherein the ECs are selected from the group consisting of umbilical vein ECs (UVECs), brain ECs, spinal cord ECs, or olfactory bulb ECs.

69. The composition of any of claims 58-68, wherein the ECs are allogeneic with respect to a subject to whom the ECs are to be administered.

70. The composition of any of claims 58-68, wherein the ECs are autologous with respect to a subject to whom the ECs are to be administered.

71. The composition of any of claims 58-70, wherein the ECs have the same MHC/HLA type as a subject to whom the cells are to be administered.

72. The composition of any of claims 58-71, wherein the ECs are mitotically inactive.

73. The composition of any of claims 58-72, wherein the ECs are differentiated ECs.

74. The composition of any of claims 58-73, wherein the ECs are adult ECs.

75. The composition of any of claims 58-74, wherein the neural cells are primary neural cells.

76. The composition of any of claims 58-75, wherein the neural cells are a cultured neural cell line.

77. The composition of any of claims 58-76, wherein the neural cells are mammalian neural cells.

78. The composition of any of claims 58-77, wherein the neural cells are primate neural cells.

79. The composition of any of claims 58-78, wherein the neural cells are human neural cells.

80. The composition of claim 77, wherein the neural cells are rabbit, rat, mouse, guinea pig, goat, pig, sheep, cow, horse, cat or dog mammalian neural cells.

81. The composition of any of claims 58-80, wherein the neural cells are selected from the group consisting of neuronal cells, glial cells, neural stem cells, neural progenitor cells, neuronal progenitor cells, glial progenitor cells.

82. The composition of any of claims 58-81, wherein the neural cells are allogeneic with respect to the subject to whom the cells are to be administered.

83. The composition of any of claims 58-81, wherein the neural cells are autologous with respect to the subject to whom the cells are to be administered.

84. The composition of any of claims 58-83, wherein the neural cells have the same MHC/HLA type as the subject to whom the cells are to be administered.

85. The composition of any of claims 58-84, wherein the neural cells are mitotically inactive.

86. The composition of any of claims 58-85, wherein the neural cells are differentiated neural cells.

87. The composition of any of claims 58-86, wherein the neural cells are adult neural cells.

88. The composition of any of claims 58-87, wherein the E4ORF1+ ECs comprise a nucleic acid molecule that encodes an adenovirus E4ORF1 polypeptide.

89. The composition of claim 88, wherein the nucleic acid molecule is in a vector.

90. The composition of claim 89, wherein the vector is a retroviral vector.

91. The composition of claim 89, wherein the retroviral vector is a lentiviral vector.

92. The composition of claim 89, wherein the retroviral vector is a Maloney murine leukemia virus (MMLV) vector.

93. The composition of any of claims 88-92, wherein the nucleic acid molecule is integrated into the genomic DNA of the EC.

94. The composition of any of claims 58-93, wherein the ratio of the E4ORF1+ ECs to the neural cells is about 1:10, or about 1:9, or about 1:8, or about 1:7, or about 1:6, or about 1:5, or about 1:4, or about 1:3, or about 1:2, or about 1:1, or about 2:1, or about 3:1, or about 4:1, or about 5:1, or about 6:1, or about 7:1, or about 8:1, or about 9:1, or about 10:1.

95. The composition of any of claims 58-94, comprising a physiological saline solution.

96. The composition of any of claims 58-95, comprising a biocompatible matrix material.
